(19) United States
(12) Patent Application Publication
(10) Pub. No.: US 2006/0079690 A1
(43) Pub. Date: Apr. 13, 2006

(54) Processes for preparing 7-hydroxy-3,4-dihydro-2(1H)-quinolinone and the use in aripiprazole preparation thereof

(76) Inventors: Vladimir Naddaka, Lod (IL); Guy Davidi, Even-Yehuda (IL); Eyal Klopfer, Tel Aviv (IL); Oded Arad, Rechovot (IL); Joseph Kaspi, Givatayim (IL)

Correspondence Address: Martin MOYNIHAN;PRTSI, Inc., P.O. Box 16446, Arlington, VA, 22215, US

(21) Appl. No.: 11/246,278

(22) Filed: Oct. 11, 2005

Related U.S. Application Data
(60) Provisional application No. 60/617,046, filed on Oct. 12, 2004.

(51) Int. Cl. C07D 215/36

(52) U.S. Cl. 546/158

(57) ABSTRACT

The present invention provides improved processes for preparing the intermediate 7-hydroxy-3,4-dihydro-2(1H)-quinolinone (7-HQ), which may be used in preparing the drug aripiprazole. Among these processes are included three efficient processes for preparing 7-hydroxy-3,4-dihydro-2(1H)-quinolinone comprising reacting N-(3-methoxyphenyl)-3-chloropropionamide with AlCl₃ using novel reaction conditions thus obtaining a substantially pure product, which may be used in the subsequent steps for obtaining aripiprazole without further purification.

### RELATED APPLICATIONS

The present application claims the benefit of U.S. Provisional Patent Application No. 60/617,046, filed on Oct. 12, 2004, which is incorporated herein by reference in its entirety.

### FIELD OF THE INVENTION

The present invention relates to improved processes for preparing 7-hydroxy-3,4-dihydro-2(1H)-quinolinone (7-HQ), which is an intermediate valuable in the synthesis of the drug aripiprazole.

### BACKGROUND OF THE INVENTION

Aripiprazole (7-{4-[4-(2,3-dichlorophenyl)-1-piperazinyl]-butoxy}-3,4-dihydro-2(1H)-quinolinone) is represented by formula (I).

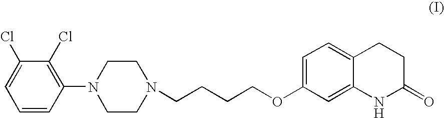

The drug is useful for treating schizophrenia and is available in tablets of different dosages.

Several synthetic methods of aripiprazole preparation are described in U.S. Pat. No. 5,006,528 including the method illustrated in scheme 1.

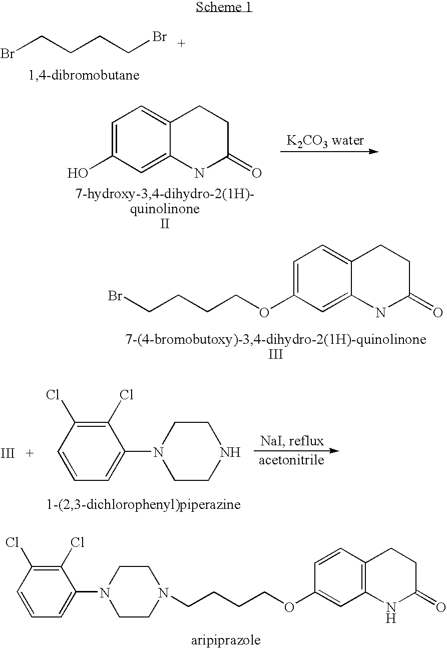

According to this synthetic method aripiprazole is prepared in two steps. The first step comprises alkylating the hydroxy group of 7-hydroxy-3,4-dihydro-2(1H)-quinolinone (hereinafter 7-HQ) of formula (II) with 1,4-dibromobutane to obtain 7-(4-bromobutoxy)-3,4-dihydroquinolinone of formula (III). A mixture of potassium carbonate, 7-HQ and 3 molar equivalents of 1,4-dibromobutane in water is refluxed for 3 hours. The reaction mixture thus obtained is extracted with dichloromethane, dried with anhydrous magnesium sulfate, and the solvent is removed by evaporation. The residue is purified by means of a silica gel column chromatography (eluent:dichloromethane), eluent evaporation and re-crystallization from a mixture of n-hexane and ethanol to obtain 7-(4-bromobutoxy)-3,4-dihydro-2(1H)-quinolinone ((III), having a melting point of 110.5-111.0° C.

In the second step the product ((III) is reacted with 1-(2,3-dichlorophenyl)piperazine to obtain aripiprazole. Thus, a suspension of ((III) and sodium iodide in acetonitrile is refluxed for 30 minutes. Triethylamine and 1-(2,3-dichlorophenyl)piperazine are added to the suspension and the reaction mixture is further refluxed for 3 hours. The solvent is then removed by evaporation, and the residue thus obtained is dissolved in chloroform, washed with water and dried with anhydrous magnesium sulfate. The solvent is removed by evaporation, and the residue is re-crystallized twice from ethanol to give aripiprazole having a melting point of 139.0-139.5° C.

7-HQ may be prepared according to the method which was reported by Mayer, F. et al, Ber. 1927, 60B, 854-64. The method comprises heating a mixture of N-(3-hydroxyphenyl)-3-chloropropionamide (hereinafter 3-HPCA) and AlCl₃ to obtain a melt, which consists of a mixture of two products: 7-HQ and 5-hydroxy-3,4-dihydro-2(1H)-quinolinone (hereinafter 5-HQ).

Scheme 2 below demonstrates this reaction.

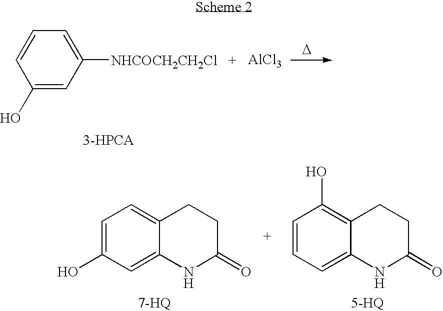

A second method of preparing 7-HQ was described by Shigematsu, N. et al, Chem. Pharm. Bull. 1961, 9, 975-5. The method comprises heating a mixture of 3-HPCA, AlCl₃, KCl, and NaCl (in ratio of 5:25:3:3 respectively) at 155-165° C. for 1 hour. Then, the reaction mixture is cooled to room temperature and quenched by pouring it into cold water.

While repeating the method developed by Shigematsu et al, it has been found by the inventors of the present invention, that after reaction completion the reaction mixture solidifies at a temperature of about 100° C. Therefore, quenching the reaction mixture in a large scale process could be very problematic. By slowly pouring the reaction mixture into water, the reaction was quenched and crude 7-HQ was precipitated in the form of a stable red-violet complex with AlCl₃. However, decomposing the complex by the traditional methods, namely by using aqueous hydrochloric acid or ice-water was difficult. It has been found also that the reaction mixture contained about 15% of 5-HQ after reaction completion. It may be therefore concluded that the 80% yield of pure colorless 7-HQ reported for the Shigematsu's method is not realistic.

This intramolecular Friedel-Crafts alkylation of 3-HPCA with AlCl₃ results in a ring closure, which occurs at an ortho position to obtain a lactam ring. Since 3-HPCA is asymmetrical there are two different ortho positions in this molecule which may lead to two different isomers upon ring closure. Owing to the deactivation of this aromatic ring towards electrophilic substitution in the conditions of the reaction (Bull. Soc. Chim. Fr. 1984, 11, 285), the reaction is sluggish and may be performed only at high temperatures (from 140° C. to 170° C.). At these conditions, the cyclization in the two ortho positions in 3-HPCA (to yield 7-HQ and 5-HQ) is inevitable. However, because formation of 7-HQ is via cyclization process, wherein the steric hindrance is low, the formation of this compound is preferable in comparison to 5-HQ.

A method of preparing 3-HPCA was also proposed by Mayer, F. et al, Ber. 1927, 60B, 854-64, by adding 3-chloropropionyl chloride dropwise to a solution of 3-aminophenol in dried acetone. It was reported also, in the above mentioned paper, that 3-HPCA may be obtained by this method in 75% yield. The reaction is demonstrated in scheme 3.

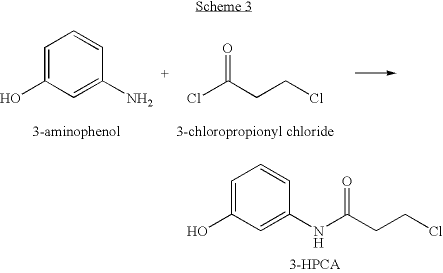

However, the problem that occurs in the presently existing methods of 3-HPCA preparation, such as the method reported by F. Mayer et al, is that the starting material 3-aminophenol is toxic and therefore its industrial use is very problematic.

In view of the limitation described above, there is an unmet need for a simple, more environmentally friendly and efficient process, which would be suitable for large scale production for preparing 7-HQ without using 3-HPCA. Thus, the industrial usage of 3-aminophenol will be avoided. The starting material N-(3-methoxyphenyl)-3-chloropropionamide (hereinafter 3-MPCA) may be used for preparing 7-HQ instead of 3-HPCA, thus avoiding the use of 3-aminophenol. The starting material for preparing 3-MPCA is m-anisidine (3-methoxyaniline).

The reaction is demonstrated in scheme 4.

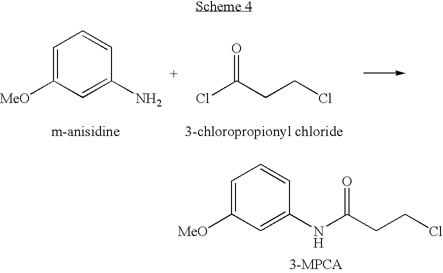

m-anisidine is considerably less toxic than 3-aminophenol and is classified only as irritant while 3-aminophenol is classified as toxic material. By choosing the starting material 3-MPCA instead of 3-HPCA in the preparation of 7-HQ, which is traditionally carried out in a melt, more convenient reaction conditions may be used.

3-MPCA may be prepared from m-anisidine using the method described by Tzeng et al. in J. Chin. Chem. Soc., 2000, 47, 155-162. According to the teaching of this article, 3-MPCA is further reacted with AlCl₃ in chlorobenzene at 120° C. to obtain a mixture of three compounds: 3-(4-chlorophenyl)propionanilide and 3-(2-chlorophenyl)propionanilide, which are the addition products of an intermolecular reaction, and 7-HQ, which is the product of intramolecular cyclization (The products were obtained in a 9/9/1 ratio respectively). Hence, 7-HQ is obtained by this process from 3-MPCA in 4% yield.

Thus, there is an additional need in the art for a method of preparing 7-HQ using 3-MPCA as starting material in high quality and higher yield than 4%. The inventors of the present invention have surprisingly uncovered that when same reaction as described by Tzeng et al is carried out in a mixture containing a high boiling point solvent instead of using chlorobenzene, the main product is 7-HQ, which is obtained in more than 60% yield having a purity equal to or greater than 99.3%, so the product 7-HQ may be used in the subsequent steps of preparing aripiprazole without further purification.

### SUMMARY OF THE INVENTION

The present invention provides improved processes for preparing 7-HQ, which may be consequently used for an improved preparation of aripiprazole by intramolecular Friedel-Crafts cyclization of 3-MPCA.

In one embodiment of the present invention 7-HQ is prepared by reacting 3-MPCA with a Lewis acid in a mixture containing a high-boiling point solvent. The usage of high-boiling point solvent enables improving the mixing ability on large scale preparations.

In another embodiment of the present invention an improved process for preparing 7-HQ is carried out by reacting 3-MPCA with a Lewis acid in the presence of a salt, which improves the stirring of the reaction mixture.

In yet another embodiment of the present invention 7-HQ is prepared by reacting 3-MPCA with a Lewis acid in the melt.

In a preferred embodiment of the present invention each one of the processes which are described herein produces highly pure 7-HQ, having purity of at least 93% (by HPLC), which may be used in the subsequent reactions for improved preparation of aripiprazole without additional purification.

### DETAILED DESCRIPTION OF THE PREFERRED EMBODIMENTS

The present invention is based on the surprising finding that, contrary to the teaching of Mayer F et al., Ber. 1927, 60B, 854-64, it is not necessary to produce 7-HQ in the melt, a process which is not easily implemented to large-scale, and instead the reaction may be carried out in an organic solvent. The reason that reactions in the melt are in many cases not suitable for scaling up is that the mixing on large scale is inefficient and low yields are obtained. On the other hand adding a solvent may greatly improve the mixing on large scale because the solvent is a diluent that reduces the viscosity of the reaction mixture.

The present invention is based also on another surprising finding that by choosing the starting material 3-MPCA instead of 3-HPCA for preparing 7-HQ, the use of the toxic reagent 3-aminophenol may be avoided.

The conversion of 3-MPCA to 7-HQ involves demethylation of the methoxy group and ring closure to produce a mixture of 7-HQ and 5-HQ.

The process is described in Scheme 5.

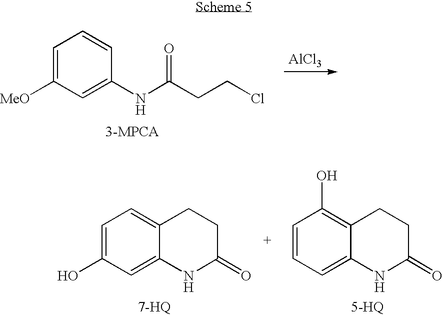

In one embodiment of the present invention, the process is based on using a Lewis acid, which may be selected from the group consisting of AlCl₃, AlBr₃, FeCl₃, FeBr₃, SbF₅, TiCl₄, SnCl₄, BF₃, SbCl₅, ZnCl₂ and the like, all known in the art to catalyze Friedel-Crafts reactions, with AlCl₃ being preferable. About 2-8 molar equivalents of AlCl₃, preferably 4-6 molar equivalents of AlCl₃, and more preferably about 5 equivalents of AlCl₃ per one mole of the starting material are used in the process for preparing 7-HQ.

In a preferred embodiment of the present invention, the process for preparing 7-HQ may be conducted using different reaction conditions.

In one embodiment of the present invention 7-HQ is prepared in a mixture containing a high-boiling point solvent; the process comprises the steps of:

a) reacting one equivalent of 3-MPCA with about 2-8 molar equivalents of AlCl₃, preferably 4-6 molar equivalents of AlCl₃, and more preferably about 5 equivalents of AlCl₃ in a mixture containing a high boiling point solvent and at a temperature of 140-220° C.;

b) quenching the reaction mixture with cold water and isolating a complex of 7-HQ with AlCl₃;

c) decomposing the complex of 7-HQ with AlCl₃ by preparing a solution in a C₁-C₄ alcohol and adding a base to produce pH of about 7;

d) isolating 7-HQ by filtration; and

e) optionally re-crystallizing 7-HQ from an organic solvent

In one aspect of the present invention the high boiling solvent may be selected, without limitation, from the group consisting of N,N-disubstituted amides, sulfoxides and sulfones, wherein such amides, sulfoxides and sulfones can be: N,N-dimethylformamide (DMF), N,N-dimethylacetamide (DMA), N,N-dimethylsulfoxide (DMSO), tetramethylene sulfone (sulfolane) and the like, high boiling point amines (boiling point of at least 160° C.), wherein such amines can be: tributylamine, tripentylamine, trihexylamine, triheptylamine, trioctylamine and the like, high boiling point ethers, wherein such ethers can be: diisoamyl ether, diglyme, triglyme and the like, high boiling point hydrocarbons like decahydronaphthalene and paraffins and the like and any mixtures thereof. Preferable diluent is DMA.

In another aspect of the present invention the amount of the diluent may be in the range of from 0.1 to 1.5 molar equivalents relative to 1 mole of 3-MPCA.

In yet another aspect of the present invention the preferable amount of the diluent is about 0.5 equivalents.

In yet another aspect of the present invention, the reaction mixture is heated under stirring to a temperature in the range of from 140° C. to 220° C., preferably from 155° C. to 165° C., after the reactor is charged with 3-MPCA and AlCl₃, while reaction mixture remains as a stirred liquid throughout the reaction, which is substantially completed during a time interval of 30 minutes to 4 hours, depending upon the reaction temperature.

In yet another aspect of the present invention, the reaction mixture is quenched by slowly pouring it into cold water after cooling to 50° C. Then, a stable red-violet complex of 7-HQ with AlCl₃ is collected by filtration. The complex contains about 2% of 5-HQ.

In yet another aspect of the present invention, decomposing the complex is carried out by dissolving it in a C₁-C₄ alcohol followed by addition of an inorganic base to produce pH of about 7.

In yet another aspect of the present invention, the C₁-C₄ alcohol, used for decomposing the complex of 7-HQ with AlCl₃, may be selected from the group consisting of methanol, ethanol, 1-propanol, and 2-propanol, or a mixture thereof. Preferable C₁-C₄ alcohol is methanol.

In yet another aspect of the present invention, the inorganic base is selected from the group consisting of sodium hydroxide, potassium hydroxide, calcium hydroxide and the like. Preferable inorganic base is sodium hydroxide.

In yet another aspect of the present invention 7-HQ is obtained by this process having a purity greater than 98.5%, preferably greater than 99% and more preferably equal or greater than 99.3%, without using chromatographic purification.

In an embodiment of the present invention 7-HQ may be re-crystallized from an organic solvent.

In another embodiment of the present invention the organic solvent that may be used for re-crystallizing 7-HQ is selected from the group consisting of methanol, ethanol, 1-propanol, 2-propanol, 1-butanol, 2-butanol and isobutanol, preferably methanol.

In another embodiment of the present invention 7-HQ is prepared in the presence of an inorganic salt, which improves the stirring of the reaction mixture; the process comprises the steps of:

a) reacting one equivalent of 3-MPCA with 2-8 equivalents of AlCl₃, preferably with 4-6 equivalents of AlCl₃ and more preferably with about 5 equivalents of AlCl₃ in the presence of an inorganic salt at a temperature of 140-220° C.;

b) quenching the reaction mixture with an aqueous solution of an inorganic acid and isolating a complex of 7-HQ with AlCl₃;

c) slurrying the complex in water in order to eliminate excess of salts from the compound;

d) decomposing the complex of 7-HQ with AlCl₃ by preparing a solution in a C₁-C₄ alcohol and adding a base to produce pH of about 7;

e) isolating 7-HQ by filtration.

In one aspect of the present invention the inorganic salt is selected from the group consisting of sodium chloride, potassium chloride, sodium bromide, potassium bromide, sodium sulfate, potassium sulfate, magnesium sulfate, or a mixture thereof. A preferable inorganic salt is sodium chloride.

In another aspect of the present invention the amount of sodium chloride used in the process varies from 0.3 g to 1.5 g relative to 1 g of 3-MPCA.

In yet another aspect of the present invention the preferable amount of sodium chloride used in the process is 0.5 g-1 g relative to 1 g of 3-MPCA.

In yet another aspect of the present invention, the reaction mixture is heated under stirring to a temperature in the range of from 140° C. to 220° C., preferably from 155° C. to 165° C., after the reactor is charged with 3-MPCA and AlCl₃, while the reaction mixture remains as a stirred slurry throughout the reaction, which is substantially completed during a time interval of 30 minutes to 4 hours, depending upon the reaction temperature.

In yet another aspect of the present invention, the reaction mixture is quenched by slowly pouring it into an aqueous solution of a cold inorganic acid after cooling to 110° C. Quenching may be also effected by slowly adding cold water to the reaction mixture cooled to 50° C. In both cases a stable red-violet complex of 7-HQ with AlCl₃, which contains about 2% of 5-HQ, is collected by filtration.

In yet another aspect of the present invention, the inorganic acid used for quenching is selected from the group consisting of hydrochloric acid, sulfuric acid, nitric acid and the like.

In yet another aspect of the present invention, the complex is slurried in water to remove the salts from the complex.

In yet another aspect of the present invention, the volume of water in the slurry ranges from 3 ml to 10 ml relative to 1 g of the complex. Preferable volume of water in the slurry is 5 ml relative to 1 g of complex.

In yet another aspect of the present invention, the temperature of the slurry is in the range of from 25° C. to 100° C., preferably about 50° C.

In yet another aspect of the present invention, the complex contains about 2% of 5-HQ after the slurrying process.

In yet another aspect of the present invention, decomposing the complex is carried out by dissolving it in a C₁-C₄ alcohol followed by addition of an inorganic base to produce pH of about 7.

In yet another aspect of the present invention, the inorganic base may be selected from the group consisting of sodium hydroxide, potassium hydroxide, calcium hydroxide and the like. Preferable inorganic base is sodium hydroxide.

In yet another aspect of the present invention the C₁-C₄ alcohol is selected from the group consisting of methanol, ethanol, 1-propanol and 2-propanol. A preferable C₁-C₄ alcohol is methanol.

In yet another aspect of the present invention 7-HQ is obtained by this process having a purity greater than 98.5%, preferably greater than 99% and more preferably equal or greater than 99.4%, without using chromatographic purification.

In another embodiment of the present invention 7-HQ is prepared in the melt; the process comprises the steps of:

a) reacting one equivalent of 3-MPCA with 2-8 equivalents of AlCl₃, preferably with 4-6 equivalents of AlCl₃ and more preferably with about 5 equivalents of AlCl₃ at a highest concentration attainable, i.e. in a melt, at a temperature ranging from 140° C. to 220° C. for a period of time sufficient to completely converting 3-MPCA to a complex of 7-HQ with AlCl₃;

b) quenching the reaction mixture with an aqueous solution of an inorganic acid and isolating a complex of 7-HQ with AlCl₃;

c) decomposing the complex of 7-HQ with AlCl₃ by preparing a solution in a C₁-C₄ alcohol and adding a base to produce pH of about 7;

d) isolating 7-HQ by filtration.

In another aspect of the present invention the C₁-C₄ alcohol used for decomposing the complex of 7-HQ with AlCl₃ is selected from methanol, ethanol, 1-propanol, and 2-propanol, or a mixture thereof, preferably methanol.

In yet another aspect of the present invention, the reaction mixture is heated under stirring to a temperature in the range of from 140° C. to 220° C., preferably from 155° C. to 165° C., after the reactor is charged with 3-MPCA and AlCl₃, while reaction mixture remains as a stirred liquid throughout the reaction, which is substantially completed during a time period of 30 minutes to 4 hours depending upon the temperature.

In yet another aspect of the present invention, the progress of the reaction may be monitored by TLC [eluent: (1:1:0.1) ethyl acetate: n-hexane: IPA; R_{∫} (7-HQ)=0.28, R_{∫} (5-HQ)=0.37, R_{∫} (3-HPCA)=0.59]. The reaction may be ceased after monitoring the disappearance of most of the starting material 3-HPCA.

In another aspect of the present invention the inorganic acid used for quenching is selected from the group consisting of hydrochloric acid, sulfuric acid, nitric acid and the like.

In yet another aspect of the present invention, the reaction mixture is quenched by slowly pouring it into cold solution of an inorganic acid, after cooling to 110° C. Quenching may be also effected by slowly adding cold water to the reaction mixture cooled to 50° C. In both cases a stable red-violet complex of 7-HQ with AlCl₃, which contains about 2% of 5-HQ, is collected by filtration.

In yet another aspect of the present invention, decomposing the complex of 7-HQ with AlCl₃ is carried out by dissolving it in a C₁-C₄ alcohol followed by addition of inorganic base to the solution to produce a pH of about 7.

In yet another aspect of the present invention, the inorganic base may be selected from the group consisting of sodium hydroxide, potassium hydroxide, calcium hydroxide and the like. Preferable inorganic base is sodium hydroxide.

In yet another aspect of the present invention, the C₁-C₄ alcohol used for decomposing the complex of 7-HQ with AlCl₃ is selected from the group consisting of methanol, ethanol, 1-propanol, and 2-propanol, or a mixture thereof. A preferable C₁-C₄ alcohol is methanol.

In yet another aspect of the present invention, 7-HQ is obtained by this process having a purity greater than 98.5%, preferably greater than 99% and more preferably equal or greater than 99.5%, without using chromatographic purification.

In a preferred embodiment of the present invention, each of the three different processes described above produce highly pure 7-HQ, which may be used in the subsequent reactions for preparing aripiprazole without additional purification.

The present invention will be concretely illustrated by examples, which show the processes for preparing 7-HQ.

While the invention will now be described in connection with certain preferred embodiments in the all alternatives, modifications and equivalents following examples so that aspects thereof may be more fully understood and appreciated, it is not intended to limit the invention to these particular embodiments. On the contrary, it is intended to cover as may be included within the scope of the invention as defined by the appended claims. Thus, the following examples which include preferred embodiments will serve to illustrate the practice of this invention, it being understood that the particulars are shown by way of example and for purposes of illustrative discussion of preferred embodiments of the present invention only and are presented in the cause of providing what is believed to be the most useful and readily understood description of formulation procedures as well as of the principles and conceptual aspects of the invention.

### EXAMPLES

Analytical measurements of the 7-HQ samples were performed using an HPLC system equipped with Phenomenex Luna C8(2) column, 5 μm, 250×4.6 mm, and a UV detector operated on 215 nm. Analyses were performed using the following mobile phase, at flow rate of 1.0 ml/minute, temperature of 30° C., and run time of 35 minutes.

Mobile phase: 80.5:19.5 (v/v) 0.05M KH₂PO₄ buffer:acetonitrile

The retention time of 7-HQ is 6.1-6.8 minutes.

### Example 1

### Preparation of 7-HQ in a Mixture Containing a High Boiling Solvent

A 3.0 l reactor was charged with 3-MPCA (300 g, 1.4 mole), AlCl₃ (920 g, 7.0 mole, 5 eq.) and N,N-dimethylacetamide (65 ml, 61 g, 0.5 eq.) and the reaction mixture was heated under stirring to about 160° C. to obtain a readily stirred liquid. The reaction mixture was held at 155-165° C. for about four hours under stirring, then cooled to about 50° C. Cold water (1500 ml) was added for a time period of half an hour and the mixture was stirred under heating to about 95° C. for one hour. The suspension thus obtained was cooled to about 50° C. and a red-violet solid was collected by filtration, washed with water (400 ml) and dried in an oven at 50° C. overnight to yield a red-violet complex of 7-HQ with AlCl₃ (202 g), containing about 2% of 5-HQ.

The complex (202 g) was dissolved in methanol (1600 ml) while heating under reflux and 47% aqueous sodium hydroxide solution was added to produce a pH of about 7. The hot solution was filtered and activated carbon (6 g) was added to the filtrate. The mixture was heated under reflux for half an hour and the activated carbon was collected by filtration. Methanol (about 1400 ml) was removed from the filtrate by evaporation and water (200 ml) was added to the residue thus obtained. The mixture was stirred at ambient temperature for half an hour and the colorless crystals were collected by filtration, washed with a cold methanol-water (1:1) mixture (10 ml) and dried at 50° C. overnight to yield 7-hydroxy-3,4-dihydro-2(1H)-quinolinone (140.5 g, 61.3% yield, having a purity of 99.3% by HPLC).

Re-crystallization from methanol gave colorless crystals: mp 230-231.5° C.

### Example 2

### Preparation of 7-HQ in the Presence of a Salt

A 0.5 l reactor was charged with 3-MPCA (40 g, 0.185 mole), AlCl₃ (125 g, 0.925 mole, 5 eq.) and anhydrous sodium chloride (20 g) and the reaction mixture was heated under stirring to about 160° C. to obtain a readily stirred slurry. The reaction mixture was held at 155-165° C. for four hours. The reaction mixture was cooled to about 50° C. and quenched by slowly adding ice cold diluted hydrochloric acid (200 ml of 5% HCl) to the reactor. The suspension thus obtained was heated to 50° C. and a red-violet solid was collected by filtration. The red-violet solid was slurried at 50° C. in water (100 ml) to remove the salts from the compound, and the solid was collected by filtration, washed with water (30 ml) and dried in an oven at 50° C. overnight to yield a red-violet complex of 7-HQ with AlCl₃ (27.1 g), containing about 2% of 5-HQ.

The complex (27.1 g) was dissolved in methanol (220 ml) while heating under reflux and 47% aqueous sodium hydroxide solution was added to produce a pH of about 7. The hot solution was filtered and activated carbon (0.8 g) was added to the filtrate. The mixture was heated under reflux for half an hour and the activated carbon was collected by filtration. Methanol (about 190 ml) was removed from the filtrate by evaporation and water (30 ml) was added to the residue thus obtained. The mixture was stirred at ambient temperature for half an hour and the colorless crystals were collected by filtration, washed with a cold methanol-water (1:1) mixture (20 ml) and dried at 50° C. overnight to yield 7-hydroxy-3,4-dihydro-2(1H)-quinolinone (18.8 g, 62.3% yield, having a purity of 99.4% by HPLC).

### Example 3

### Preparation of 7-HQ in a Melt

A 2.0 l reactor was charged with 3-MPCA (150 g, 0.69 mole) and AlCl₃ (460 g, 3.45 mole, 5 eq.).The reaction mixture was heated under stirring to about 160° C. to obtain a liquid. The reaction mixture was stirred and held at 155-165° C. for about four hours. Stirring was stopped and the reaction mixture was cooled to 50° C. Ice cold diluted hydrochloric acid (750 ml of 5% HCl) was added to the reactor during half an hour and the mixture was stirred while heating to about 95° C. for one hour. The suspension thus obtained was cooled to about 50° C. and a red-violet solid was collected by filtration, washed with water (200 ml) and dried in an oven at 50° C. overnight to yield the red-violet complex of 7-HQ with AlCl₃ (100 g), containing about 2% of 5-HQ.

The complex (100 g) was dissolved in methanol (800 ml) while heating under reflux and 47% aqueous sodium hydroxide solution was added to produce a pH of about 7. The hot solution was filtered and activated carbon (3 g) was added to the filtrate. The mixture was heated under reflux for half an hour and the activated carbon was collected by filtration. Methanol (about 700 ml) was removed from the filtrate by evaporation and water (100 ml) was added to the residue thus obtained. The mixture was stirred at ambient temperature for half an hour and the colorless crystals were collected by filtration, washed with a cold methanol-water (1:1) mixture (50 ml) and dried at 50° C. overnight to yield 7-hydroxy-3,4-dihydro-2(1H)-quinolinone (70.2 g, 61.3% yield, having a purity of 99.5%by HPLC).

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

What is claimed is:

1. An improved process for preparing 7-hydroxy-3,4-dihydroquinolinone (7-HQ) by reacting N-(3-methoxyphenyl)-3-chloropropionamide (3-MPCA) with a Lewis acid, wherein 7-HQ is obtained having a purity greater than 98.5%, preferably greater than 99% and more preferably equal to or greater than 99.3%, without using chromatographic purification.

2. The process according to claim 1, wherein the said Lewis acid is selected from the group consisting of AlCl₃, AlBr₃, FeCl₃, FeBr₃, SbF₅, TiCl₄, SnCl₄, BF₃, SbCl₅, ZnCl₂ and the like.

3. The process according to claim 2, wherein the said Lewis acid is AlCl₃.

4. A process for preparing 7-hydroxy-3,4-dihydroquinolinone (7-HQ), according to claim 3, in a mixture containing a high-boiling point solvent, the process comprising:
a) reacting one equivalent of 3-MPCA with 2-8 equivalents of AlCl₃, preferably with 4-6 equivalents of AlCl₃ and more preferably with about 5 equivalents of AlCl₃ in a mixture containing a high boiling point solvent at a temperature of 140-220° C.;
b) quenching the reaction mixture with cold water and isolating a complex of 7-HQ with AlCl₃;
c) decomposing the complex of 7-HQ with AlCl₃ by preparing a solution in a C₁-C₄ alcohol and adding a base to produce pH of about 7;
d) isolating 7-HQ by filtration; and
e) optionally re-crystallizing 7-HQ from an organic solvent.

5. The process according to claim 4, wherein the solvent is selected from the group consisting of N,N-disubstituted amides, sulfoxides and sulfones, wherein such amides, sulfoxides and sulfones are: N,N-dimethylformamide (DMF), N,N-dimethylacetamide (DMA), N,N-dimethylsulfoxide (DMSO), tetramethylene sulfone (sulfolane) and the like, high boiling point amines (boiling point of least 160° C.), wherein such amines are: tributylamine, tripentylamine, trihexylamine, triheptylamine, trioctylamine and the like, high boiling point ethers, wherein such ethers can be: diisoamyl ether, diglyme, triglyme and the like, high boiling point hydrocarbons like decahydronaphthalene and paraffins and the like, and mixtures thereof.

6. The process according to claim 5, wherein the solvent is N,N-dimethylacetamide (DMA).

7. The process according to claim 4, wherein said C₁-C₄ alcohol used for decomposing the complex of 7-HQ and AlCl₃ is selected from the group consisting of methanol, ethanol, 1-propanol, 2-propanol, and a mixture thereof.

8. The process according to claim 7, wherein said alcohol is methanol.

9. The process according to claim 4, wherein the solvent used for crystallizing 7-HQ is selected from the group consisting of methanol, ethanol, 1-propanol, 2-propanol, 1-butanol, 2-butanol, isobutanol, and a mixture thereof.

10. The process according to claim 9, wherein the solvent is methanol.

11. The process according to claim 4, wherein the yield of the obtained 7-HQ by reaction of 3-MPCA with AlCl₃ in a mixture with high boiling point solvent is greater than 10%.

12. An improved process for preparing 7-hydroxy-3,4-dihydroquinolinone (7-HQ) in the presence of an inorganic salt; the process comprising:
a) reacting one equivalent of 3-MPCA with 2-8 equivalents of AlCl₃, preferably with 4-6 equivalents of AlCl₃ and more preferably with about 5 equivalents of AlCl₃ in the presence of an inorganic salt at a temperature of 140-220° C.;
b) quenching the reaction mixture with aqueous solution of an inorganic acid and isolating a complex of 7-HQ with AlCl₃;
c) slurrying the complex in water in order to eliminate excess of salts from the compound;
d) decomposing the complex of 7-HQ with AlCl₃ by preparing a solution in a C₁-C₄ alcohol and adding a base to produce pH of about 7; and
e) isolating 7-HQ by filtration.

13. The process according to claim 12, wherein said inorganic salt is selected from the group consisting of NaCl, KCl, NaBr, KBr, Na₂SO₄, K₂SO₄, MgSO₄ and the like, and combinations thereof.

14. The process according to claim 13, wherein said inorganic salt is NaCl.

15. The process according to claim 12, wherein the said inorganic acid used for quenching is selected from the group consisting of hydrochloric acid, sulfuric acid, nitric acid and the like.

16. The process according to claim 12, wherein said C₁-C₄ alcohol used for decomposing the complex of 7-HQ and AlCl₃ is selected from the group consisting of methanol, ethanol, 1-propanol, 2-propanol, and mixtures thereof.

17. The process according to claim 16, wherein said alcohol is methanol.

18. An improved process for preparing 7-hydroxy-3,4-dihydroquinolinone (7-HQ) in a melt; the process comprising:
a) reacting one equivalent of 3-MPCA with 2-8 equivalents of AlCl₃, preferably with 4-6 equivalents of AlCl₃ and more preferably with about 5 equivalents of AlCl₃ at a highest concentration attainable, i.e. in a melt, at a temperature ranging from 140° C. to 220° C. for a period of time sufficient to completely converting 3-MPCA to a complex of 7-HQ with AlCl₃;
b) quenching the reaction mixture with aqueous solution of an inorganic acid and isolating a complex of 7-HQ with AlCl₃;
c) decomposing the complex of 7-HQ with AlCl₃ by preparing a solution in a C₁-C₄ alcohol and adding a base to produce pH of about 7; and
d) isolating 7-HQ by filtration.

19. The process according to claim 18, wherein the said inorganic acid used for quenching is selected from the group consisting of hydrochloric acid, sulfuric acid, nitric acid and the like.

20. The process according to claim 18, wherein said Cl -C₄ alcohol used for decomposing the complex of 7-HQ and AlCl₃ is selected from the group consisting of methanol, ethanol, 1-propanol, 2-propanol, and mixtures thereof.

21. The process according to claim 20, wherein said alcohol is methanol.

22. A process of preparing aripiprazole (7-{4-[4-(2,3-dichlorophenyl)-1-piperazinyl]-butoxy}-3,4-dihydro-2(1H)-quinolinone) or any pharmaceutically acceptable salt thereof, by converting 7-HQ, prepared essentially as described herein, to said aripiprazole.
